(19) United States
(12) Plant Patent Application Publication
(10) Pub. No.: US 2002/0184684 P1
(43) Pub. Date: Dec. 5, 2002

(54) Chrysanthemum plant named 'Gedi One Nov'

(76) Inventor: Dirk Pieters, Oostnieuwkerke (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,960

(22) Filed: Jun. 4, 2001

(51) Int. Cl. A01H 5/00

(52) U.S. Cl. PLT/290

(57) ABSTRACT

A new and distinct Chrysanthemum plant variety characterized by a large decorative type inflorescence, consistent flowering response to short days, blooming consistently after 49 days of short day length, free branching habit, bicolor blooms, very uniform round growth habit, and a large quantity of blooms per flowering branch.

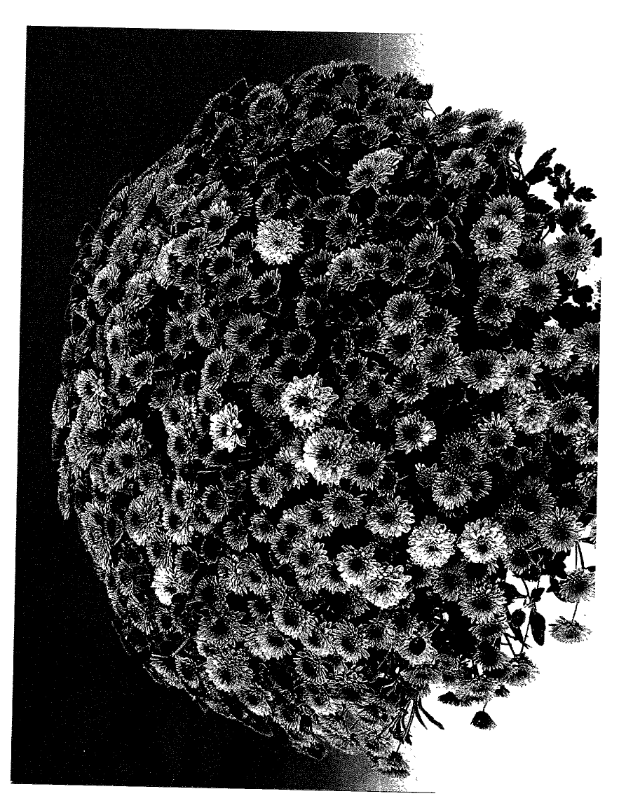

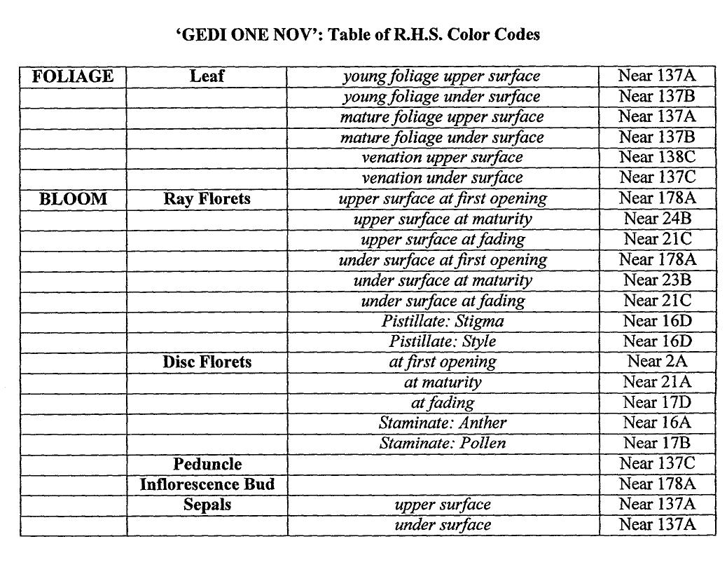

### BACKGROUND OF THE INVENTION

The present invention relates to a new and distinct cultivar of Chrysanthemum plant, botanically known as Dendranthema morifolium and herein after referred to by the cultivar name ‘GEDI ONE NOV.’

This new cultivar was the result of hybridization of a female parent, ‘Bronia’(unpatented in the United States) with a male parent, ‘Gedi OR08’ (U.S. Plant Pat. No. 11,569). The new cultivar was selected in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE NOV’ has not been observed under all possible environmental conditions. The phenotype may vary somewhat with variations in environment such as temperature, daylength, and light intensity, without, however, any variance in genotype.

The following traits have been repeatedly observe and are determined to be the unique characteristics of ‘GEDI ONE NOV.’ These characteristics in combination distinguish ‘GEDI ONE NOV’ as a new and distinct Chrysanthemum:

1. Large decorative type inflorescence;

2. Consistent flowering response to short days, blooming consistently after 49 days of short day length;

3. Free branching habit;

4. Bicolor blooms;

5. Very uniform round growth habit; and

6. Large quantity of blooms per flowering branch.

Plants of the new Chrysanthemum are similar to plants of the parent cultivars in most horticultural characteristics, however plants of the new cultivar differ from plants of the female parent cultivar ‘Bronia’ in the following characteristics:

1. The new cultivar naturally blooms 2 weeks earlier;

2. The new cultivar has different color ray florets; and

3. The new cultivar has larger diameter flowers.

The new cultivar also differed from the male parent ‘Gedi OR08’ in the following characteristics:

1. The new cultivar has an different ray floret color;

2. The new cultivar has a different flower type;

3. The new cultivar naturally blooms 2 weeks earlier; and

4. The new cultivar has larger diameter blooms.

The new cultivar can be compared to the Chrysanthemum cultivar ‘Aldo’ (disclosed in Plant Pat. No. 10,216). However plants of the new Chrysanthemum differed from the plants of the cultivar ‘Aldo’ in the following characteristics:

1. New cultivar has larger diameter blooms;

2. New cultivar has more ray florets;

3. New cultivar has a more round plant shape and greater branching; and

4. New cultivar has more blooms per flowering branch.

### BRIEF DESCRIPTION OF THE PHOTOGRAPHS

The photograph shows a plant of ‘GEDI ONE NOV’ grown in a 2 gallon container. One cutting was used in the pot, planted mid-May and grown under outdoor field conditions.

### DETAILED BITANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE NOV’

Commercial classification: Garden-type Chrysanthemum

Parentage: Hybridization of female parent ‘Bronia’ and male parent ‘Gedi OR08’

### PROPAGATION

Time to Rooting: 7 to 14 days at approximately 21° C.

Rooting habit: Fine, fibrous

### PLANT

Growth Habit: Mounding herbaceous perennial

Height: Approximately 28 cm

Growth rate: Moderate

Branching Characteristics: Free Branching

Length of Lateral Branches: Approximately 23 cm

Number of leaves per lateral branch: Approximately 24

### FOLIAGE

Leaf:

Arrangement.—Alternate single.

Average Length.—Approximately 7.6 cm.

Average Width.—Approximately 4.5 cm.

Shape of blade.—Ovate.

Apex.—Acuminate.

Base.—Attenuate.

Attachment.—Stalked.

Margin.—Serrate.

Texture of top surface.—Lightly pubescent.

Texture of bottom surface.—Lightly pubescent.

Color: young foliage upper side: Near 137A young foliage under side: Near 137B mature foliage upper side: Near 137A mature foliage under side: Near 137B venation upper side: Near 138C. venation underside: Near 137C.

Petiole:

Average Length.—Approximately 2.9 cm.

### BLOOM

Inflorescence:

Flowering habit.—Induced by darkness period greater than 13.5 hours, approximately 49 days of appropriate day length required to induce and develop blooms.

Inflorescence form.—Decorative.

Natural flowering season.—Approximately the first two weeks of September.

Number of inflorescences per stem.—Approximately 14.

Inflorescence diameter.—Approximately 6.5 cm.

Inflorescence depth.—2.1 cm.

Inflorescence longevity on plant.—Approximately 24-28 days.

Persistence.—Persistent.

Ray Florets.—.

Appearance.—Matte.

Texture.—Smooth.

Average Number perflower.—280.

Shape.—Oblanceolate.

Margin.—Entire.

Apex.—Obtuse.

Length.—Approximately 3.8 cm.

Width.—Approximately 1.1 cm

Color.—. upper surface at first opening: Near 178A upper surface at maturity: Near 24B upper surface at fading: Near 21C under surface atfirst opening: Near 178A under surface at maturity: Near 23B under surface atfading: Near 21C.

Disc florets:

Appearance.—Shiny.

Texture.—Smooth.

Average number perflower.—Approximately 30.

Shape.—Cylindric.

Apex.—Obtuse.

Average Length.—Approximately 1 cm.

Average Width.—Approximately 0.1 cm.

Color.—. at first opening: Near 2A at maturity: Near 21A atfading: Near 17D

Peduncle:

Length.—. at terminal end (shortest): Approximately 7 cm. at lateral end (longest): Approximately 12 cm.

Strength.—Moderate.

Color.—Near 137C.

Habit.—Erect.

Inflorescence bud:

Length.—Approximately 1 cm.

Diameter.—Approximately 1 cm.

Form.—Globular.

Color.—Near 178A.

Sepals:

Appearance.—Matte.

Texture.—Lightly pubescent.

Number.—Approximately 30 to 40.

Shape.—Ovate.

Margin.—Entire.

Apex.—Acute.

Color.—. upper side: Near 137A. underside: Near 137A.

### REPRODUCTIVE ORGANS

Ray Florets:

Pistillate.—Number of pistils per flower: 1 Stigma shape: 2 branched Stigma color: Near 16D Style color: Near 16D Style length: Approximately 0.6 cm.

Disc Florets:

Pistillate.—Number of pistils per flower: 1 Stigma shape: Cylindric Style length: 0.5 cm.

Staminate.—Number of stamens per flower: Approximately 5 Anther shape: Tubular Anther color: Near 16A . Pollen color: Near 17B.

### OTHER CHARACTERISTICS

Disease Resistance: Neither resistance nor susceptibility observed

What is claimed is:

1. A new and distinct variety of Chrysanthemum plant named ‘GEDI ONE NOV’ as herein illustrated and described.
